(19) United States
(12) Patent Application Publication
(10) Pub. No.: US 2022/0251623 A1
(43) Pub. Date: Aug. 11, 2022

(54) Method and apparatus for avoiding false positive coliform testing

(71) Applicant: Jonathan N. Roth, Goshen, IN (US)

(72) Inventor: Jonathan N. Roth, Goshen, IN (US)

(21) Appl. No.: 17/300,020

(22) Filed: Feb. 9, 2021

(51) Int. Cl. C12Q 1/10

(52) U.S. Cl. CPC C12Q 1/10; G01N 2333/245

(57) ABSTRACT

Coliform detection is provided via the alpha (“α”) form of enzyme substrates. The α form can be mixed with a dry growth media suitable for target Coliforms and then applied as needed to a test card format, or then mixed with water and sterilized for use a broth applied to a membrane filter detection device, for example.

### BACKGROUND OF THE INVENTION

The present invention relates to methods and apparatus for testing to determine the presence of Coliform bacteria and other microbial items in food and/or water, and more particularly, to qualitative and quantitative detection of coliforms, including Escherichia coli (“E. coli”) present in water or other test sample materials.

A number of commercially available products are used to detect the presence of various Coliforms via visual indicia (such as discriminating colors detectable under visible and/or ultra violet light), including Coliscan™, Colichrome™, and Colilert™. These products are satisfactory for many purposes. However, due to the concurrent presence of non-target micro-organisms, such as Aeromonas spp, Pseudomonas spp, false positive test results can arise when testing for Coliforms with these prior products. Briefly, the false positive test results arise because these products use, for example, the beta (“β”) form of enzyme substrates in the test medium which Aeromonas respond to in a similar manner as the Coliform being sought. For example, where β-galactoside is used as the marker for Coliforms, Aeromonas spp can be present and provide a nearly identical chromogenic or fluorogenic response. This results in the test sample being interpreted to have a higher concentration of Coliforms than is actually the case, a false positive indication.

### OBJECTIVES OF THE INVENTION

Accordingly, a primary objective of the invention is to provide improved methods and apparatus for detection of Coliforms. These improvements include providing such detection methods which:
a. are less inexpensive to manufacture and use,
b. facilitate and/or more accurate rapid detection of the micro-organisms,
c. are applicable to a wide variety of pre-existing detections apparatus,
d. accommodate a variety of different pre-existing growth and counting devices, and
e. increase user convenience and safety.

### SUMMARY OF THE INVENTION

These and other objectives of the present invention are achieved by the provision of Coliform (including E. coli) detection via incorporation of the alpha (“α”) form of enzyme substrates. The α form can be mixed with a dry growth medium, consisting of a mixture of nutrients suitable for a given target Coliform and then applied to a test card format, or mixed with water and sterilized for use as a broth applied to a standard membrane filter detection device, for example.

Other objects, advantages, and novel features of the present invention will become readily apparent from the following drawings and detailed description of certain preferred and alternative embodiments.

### BRIEF DESCRIPTION OF THE DRAWING

FIG. 1 shows schematically the method of the present invention according to a preferred embodiment.

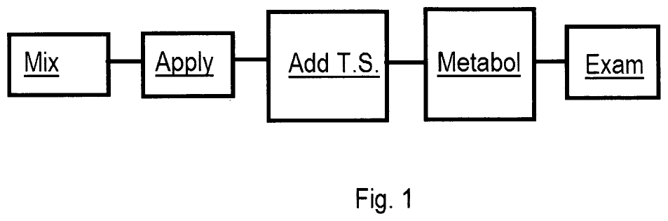

### DETAILED DESCRIPTION OF PREFERRED EMBODIMENTS

The present invention provides more accurate Coliform detection by using the alpha form of enzyme substrates as the color detection marker rather that the beta form of enzyme substrates. The present invention has applicability to a wide range of micro-organisms and a wide range of enzyme substrates. In general, by “enzyme substrate,” this specification refers to enzyme substrate substances where the β form of that material will produce a a distinct and similar color marker from the presence of a Coliform and/an Aeromonas, but the α form of that substance will produce a distinct color marker from only the presence of the Coliform or the color marker or appearance for the Aeromonas is distinct and different by being either too faint or a significantly different color. By “color,” this specification refers to a visually detectable indicia under either visible light or ultra-violet light. Thus, the present invention is applicable to both chromogenic, fluorogenic, and DUOGEN™ enzyme substrate compounds.

A specific embodiment of the present invention would, for example, use 6-chloro-3-indoxyl-α-D-galactopyranoside (or, synonymously, 6-chloro-3-indolyl-α-D-galactoside) as the enzyme substrate, commonly called “Salmon α-Galactoside.” This is commercially available from Inalco Pharmaceuticals (www.inalcopharm.com), for example, as part number CAS: 198402-61-8. This gives a pink/red color marker to indicate the Coliform bacteria.

An alternative embodiment of the present invention would, for example, use 5-Bromo-4-Chloro-3-indoxyl-α-D-galactopyranoside as the enzyme substrate, commonly called “X-α-galactoside.” This is commercially available from Inalco Pharmaceuticals (www.inalcopharm.com), for example, as part number CAS: 107021-38-8. This gives a green color marker to indicate the Coliforms.

The method of the present invention in its simplest form is to first contact an aqueous or fluidic test sample of the suspected or targeted Coliform material to the a form enzyme substrate, then allow the test sample to interact with that enzyme substrate so as to reveal the color change. In preferred embodiments, the enzyme substrate is first mixed with a growth medium, so that the test sample can more readily metabolize the enzyme substrate and provide the color marker. Use of various nutrients as the growth medium for chromogenic testing is well known, and in general, the nutrients selected in a given application will be one or more which the target microbes greatly prefer over any other nutrients, and also, for which other microbes likely to be in the test sample have little or no preferences, and cannot significantly assimilate. Thus, only the target microbes in the test sample can result in sufficient metabolism of the nutrient to cause the color or other characteristic change in the test sample.

For example, the following items can be combined to form a suitable growth medium for use with the present invention in detecting Coliforms:

| | | |
|---|---|---|
| Peptone | 4 | gm |
| Yeast extract | 2 | gm |
| Dipotassium phosphate | 3 | gm |
| Monopotassium dihydrogen phosphate | 1 | gm |
| Sodium chloride | 3 | gm |
| Sodium lauryl sulfate | 200 | mg |
| Deoxycholic Acid | 75 | mg |
| IPTG | 100 | mg |

When used with Salmon α-Galactoside, 100 mg of that enzyme substrate is added. When used alternatively with X-α-galactoside, 70 mg of the enzyme substrate is added. In alternative embodiments, this or similar formulations may incorporate alternate ingredients, so as to result in the addition and/or visualization of other than Coliform bacteria.

These ingredients can be mixed with one liter of de-ionized water and sterilized to form a broth which is then dispensed onto an absorbent pad, or the like, in a test container, such as Petri dish. The test sample is then passed through a micropore filter and then applied to the absorbent pad containing the medium, and the target microbes are allowed to grow and metabolize the growth medium and enzyme substrate to reveal the color marker. Alternatively, these ingredients can be mixed dry with 30 grams of a gum material, such as a guar gun, for powder application to a test container or surface, such as a sample card. One such suitable test card would be of the type used for R-Cards, commercially available from Roth Bioscience.

With reference to the drawing:
“Mix” refers to the step of combining the alpha form of the enzyme substrate with the growth medium,
“Apply” refers to the step of putting the combined mixture onto a card, test container, or the like in liquid or dry form,
“Add T.S”. refers to the step of putting the fluidic test sample onto or into the test container in a conventional manner,
“Metabol” refers to the step of allowing the test sample to interact with the enzyme substrate sufficient to produce the marker color, under visible and/or fluorescent light.
“Exam” refers to the step of examining the test results, either manually or via an optical scanning device or the like, to determine the presence, density, and/or abundance of the marker colors, indicative of the amount of the target coliform or microbial element which was present in the test sample.
   These steps are preferably performed in sequence, from left to right in the drawing.

Although the present invention has been shown and described herein with respect to certain preferred embodiments and alternative configurations, those were by way of illustration and example only. For example, in especially preferred embodiments, the method can include the additional steps of including an inhibitor chemical or an antibiotic in the Mix step which restricts growth of certain microbes which could confuse the test results or otherwise reduce the optimum results of the interaction of the target coliform with the alpha form of the enzyme substrate.

Also, the present invention can be used to concurrently detect the presence of Aeromonas elements in the test same, as where the alpha form of the enzyme substrate selected produces a distinct color marker for the Aeromonas, which is noticeably difference from the Coliform color marker. In addition, certain embodiments of the present invention can inhibit production of a color marker for Aeromonas spp., and instead suppress such color marker from developing as the Aeromonas spp. grows (this is in contrast with the color marker being produced with the Aeromonas growth when a beta-gal enzyme substrate would be used).

Further, the present invention can be used to quantify the reliability of conventional coliform test methods and apparatus by running those tests in parallel with the method and apparatus of the present invention. Accordingly, the spirit and scope of the present invention is intended to be limited only by the terms of the appended claims.

What is claimed is:

1. A method of detecting Coliforms comprising the steps of:
applying an alpha form of an enzyme substrate to a test container,
then depositing a fluidic test sample onto said test container,
then allowing said test sample to interact with said enzyme substrate sufficiently to create a distinct color maker, and
then examining said test container to detect the color maker and thereby determine the presence or absence or relative quantities of the Coliforms.

2. The method according to claim 1 wherein said alpha from of the enzyme substrate is first selected to have distinct color makers for the presence of Coliforms and Aeromonas elements, and then that enzyme substrate is applied to the test container.

3. The method according to claim 1 wherein prior to applying the alpha form of an enzyme substrate to a test container, that enzyme is mixed with a medium to increase growth of the target Coliforms.

4. The method according to claim 3 wherein said alpha form of the enzyme substrate is selected to be 6-chloro-3-indoxyl-α-D-galactopyranoside or a chemically equivalent alpha enzyme substrate with respect to production of Coliform color markers.

5. The method according to claim 4 wherein the Coliform to be tested for is E. coli.

6. The method according to claim 4 wherein said test container is a card, and in said step of combining the enzyme substrate with a growth medium, and one or more gums are also mixed with those components.

7. The method according to claim 3 wherein said alpha form of the enzyme substrate is selected to be 5-Bromo-4-Chloro-3-indoxyl-α-D-galactopyranoside or a chemically equivalent compound with respect to production of Coliform color markers.

8. The method according to claim 6 wherein the Coliform to be tested for is E. coli.

9. The method according to claim 1 wherein said enzyme substrate includes therewith a medium to increase growth of the target Coliforms.

10. The method of creating test cards for detection of Coliforms, comprising the steps of:
mixing an alpha form of an enzyme substrate with a growth medium and a gum to allow adherence to the card, and then
applying that mixture to the card as a dry powder.
